(19) United States
(12) Patent Application Publication
(10) Pub. No.: US 2005/0226873 A1
(43) Pub. Date: Oct. 13, 2005

(54) Medical devices and methods for modulating cell adhesion

(76) Inventors: Lucian V. Del Priore, Basking Ridge, NJ (US); Barbara J. McLaughlin, Omaha, NE (US); Henry J. Kaplan, Louisville, KY (US); Nalini S. Bora, Louisville, KY (US)

Correspondence Address: Leslie Gladstone Restaino;Brown Raysman Millstein Felder & Steiner LLP, 163 Madison Avenue, P.O. Box 1989, Morristown, NJ, 07962-1989, US

(21) Appl. No.: 10/943,728

(22) Filed: Sep. 17, 2004

Related U.S. Application Data
(60) Provisional application No. 60/507,636, filed on Sep. 30, 2003.

(51) Int. Cl. A61K 39/395

(52) U.S. Cl. 424/144.1; 424/423

(57) ABSTRACT

The present invention provides a medical device for use in inhibiting adhesion of cells. The medical device has a coating comprising a CD46 inhibitor. In one embodiment, the coating further comprises an inhibitor of β integrin. Also provided is a use of the medical device in a method for inhibiting adhesion of cells. The present invention further provides methods for inhibiting cell-substrate and cell-cell adhesion. Also provided are uses of CD46 inhibitors in these methods. Additionally, the present invention provides methods for promoting cell-substrate and cell-cell adhesion.

### RELATED APPLICATIONS

This application claims the benefit of U.S. Provisional Application Ser. No. 60/507,636, filed on Sep. 30, 2003, and entitled “Medical Devices and Methods for Modulating Cell Adhesion.” the contents of which are hereby incorporated by reference herein.

### STATEMENT OF GOVERNMENT INTEREST

This invention was made with government Support under National Eye Institute Grant No. EY09730 (NSB). As such, the United States government has certain rights in this invention.

### BACKGROUND OF THE INVENTION

Evidence is growing that the complement system may play a significant, but currently undefined, role in age-related macular degeneration (ARMD), the leading cause of blindness in the elderly population. One of the hallmarks of this disease is the formation of extracellular deposits, or drusen, between the retinal pigment epithelium (RPE), its basement membrane, and the remaining Bruch's membrane. Progression of the disease leads to RPE dysfunction, detachment, and, eventually, degeneration, which adversely affects the sensory photoreceptors and results in visual loss. Based upon immunolocalization of terminal complement complexes in drusen, some investigators have suggested that the formation of drusen involves complement activation, and that a dysfunctional condition in the RPE is an initiating event in ARMD (Johnson et al., Complement activation and inflammatory processes in drusen formation and age related macular degeneration. Exp. Eye Res., 73:887-96, 2001). Although some components of the complement system are present in the retina (Bora et al., Differential expression of the complement regulatory proteins in the human eye. Invest. Ophthalmol. Vis. Sci. 34:3576-84, 1993) and RPE (Elner et al., Immunophagocytic properties of retinal pigment epithelium cells. Science, 211:74-76, 1981), very little is known about the roles of these components—apart from the fact that they serve a protective function in innate immunity.

CD46, or membrane cofactor protein (MCP), acts as a serum protease cofactor; it degrades C3b, and prevents activation of the complement cascade, thereby protecting a host cell against autologous attack (Seya and Atkinson, Functional properties of membrane cofactor protein of complement. Biochem. J., 264:581-88, 1989; Liszewski et al., Control of the complement system. Adv. Immunol., 61:201-83, 1996; Liszewski et al., Membrane cofactor protein (MCP or CD46): newest member of the regulators of complement activation gene cluster. Annu. Rev. Immunol., 9:43-55, 1991). CD46 is a transmembrane glycoprotein that is present on most nucleated cells. It serves as a receptor for measles virus, and for C3b, C4b, and two other human pathogens (Dorig et al., The human CD46 molecule is a receptor for measles virus (Edmonston strain). Cell, 75:2953-05, 1993; Naniche et al., Human membrane cofactor protein (CD46) acts as a cellular receptor for measles virus. J. Virol., 67:6025-32, 1993; Manchester et al., Multiple isoforms of CD46 (membrane cofactor protein) serve as receptors for measles virus. Proc. Natl. Acad. Sci. USA, 91:2161-65, 1994; Dorig et al., CD46, a primate-specific receptor for measles virus. Trends Microbiol., 2:312-18, 1994; Okada et al., Membrane cofactor protein (MCP; CD46) is a keratinocyte receptor for the M protein of group A streptococcus. Proc. Natl. Acad. Sci. USA, 92:2489-93, 1995; Kallstrom et al., Membrane cofactor protein (MCP; CD46) is a cellular pilus receptor for pathogenic. Neisseria Mol. Microbiol., 25:639-47, 1997). In addition, CD46 is polarized on the basolateral membrane of epithelial cells from non-ocular tissue (Maisner et al., Two different cytoplasmic tails direct isoforms of the membrane cofactor protein (CD46) to the basolateral surface of Madin-Darby canine kidney cells. J. Biol. Chem., 271:18853-58, 1996; Maisner et al., Membrane cofactor protein (CD46) is a basolateral protein that is not endocytosed: importance of the tetrapeptide FTSL at the carboxyl terminus. J. Biol. Chem., 272:20793-99, 1997; Teuchert et al., Importance of the carboxyl-terminal FTSL motif of membrane cofactor protein for basolateral sorting and endocytosis: positive and negative modulation by signals inside and outside the cytoplasmic tail. J. Biol. Chem., 274:19979-84, 1999; Ludford-Menting et al., A functional interaction between CD46 and DLG4: a role for DLG4 in epithelial polarization. J. Biol. Chem., 277:4477-84, 2002), and is highly expressed at the blood-brain barrier (Shusta et al., Subtractive expression cloning reveals high expression of CD46 at the blood-brain barrier. J. Neuropathol. Exp. Neurol., 61:5976-04, 2002).

### SUMMARY OF THE INVENTION

In one aspect, the present invention provides a medical device for use in inhibiting adhesion of cells, wherein the medical device has a coating comprising an inhibitor of CD46. In one embodiment, the coating further comprises an inhibitor of β integrin. Also provided is a use of a medical device in a method for inhibiting adhesion of cells, wherein the medical device has a coating comprising an inhibitor of CD46.

In another aspect, the present invention provides a method for inhibiting adhesion of a cell to a substrate, by inhibiting CD46 in the cell. In one embodiment, the method further comprises the step of inhibiting β integrin in the cell. Also provided is a use of a CD46 inhibitor in a method for inhibiting adhesion of a cell to a substrate.

In still another aspect, the present invention provides a method for inhibiting cell-cell adhesion between or among non-microbial, non-leukocyte cells in a subject, by inhibiting CD46 in the subject. In one embodiment, the method further comprises the step of inhibiting β integrin in the subject. Also provided is a use of a CD46 inhibitor in a method for inhibiting cell-cell adhesion between or among non-microbial, non-leukocyte cells in a subject.

In yet another aspect, the present invention provides a method for promoting adhesion of a cell to a substrate, by increasing CD46 in the cell. In one embodiment, the method further comprises increasing β integrin in the cell.

In a further aspect, the present invention provides a method for promoting cell-cell adhesion between or among non-microbial, non-leukocyte cells in a subject, by increasing CD46 in the subject. In one embodiment, the method further comprises increasing β integrin in the subject.

Additional aspects of the present invention will be apparent in view of the description which follows.

### BRIEF DESCRIPTION OF THE FIGURES

FIG. 1 sets forth histologic sections of donor eyes, upper panel: In a donor eye immunostained with anti-CD46, staining was present on the basolateral membrane surface of the RPE. The inset in the top-right corner represents a higher magnification of basolateral staining, lower panel: Control sections showed little or no specific staining, bar=20 μm

FIG. 2 depicts confocal microscopy of horizontal sections through RPE from a human donor eye, flat-mounted and immunolabeled for CD46. (A, B) Sections from the same apical RPE plane show the absence of staining for CD46 (A), and the absence of autofluorescent pigment granules (B). (C, D) Sections from the same basolateral RPE plane show membrane staining for CD46 (C), and autofluorescence due to pigment granules in the basal RPE cytoplasm (D), bar=20 μm

FIG. 3 depicts forth confocal microscopy of horizontal sections through the RPE monolayer from primary cultures. The sections show the absence of labeling for CD46 (left) and β1 integrin (right) on the apical plane (top two panels), and the presence of basolateral membrane staining only in the basal plane (middle two panels) of the monolayer. Polarized labeling on the basal RPE surface was clearly visible in vertical sections (lower two panels), bar=20 μm

FIG. 4 depicts confocal microscopy of horizontal sections through the ARPE19 cell monolayer. The sections show the same absence of apical labeling (top two panels) and intense basolateral labeling (middle two panels) for both CD46 (left) and β1 integrin (right) in both horizontal (top two panels and middle two panels) and vertical (lower two panels) sections, bar=20 μm

FIG. 5 sets forth immunoblots of RPE from donor eyes (left) and ARPE19 cell lines (right) labeled with antibodies to CD46. Two bands are present in each lane—a dense upper band at 65 kDa and a lighter band at 55 kDa. These correspond to the heavier and lighter isoforms of CD46, respectively.

FIG. 6 illustrates an RT-PCR of RPE from donor eyes and the ARPE19 cell line. Lanes 1-4 show controls without reverse transcriptase; lanes 5-7 show PCR products of RPE harvested from donor eyes; and lane 8 shows PCR product from ARPE19 cells. All were within the expected base-pair range (448 bp) for CD46.

FIG. 7 illustrates CD46 co-immunoprecipitation with β1 integrin from RPE cell lysates from donor eyes. The left panels (A) show immunoprecipitated proteins when anti-CD46 was used as the precipitating antibody. A dense band of the CD46 protein was labeled with anti-CD46 at the expected range of 55-65 kDa, and a lighter band was labeled with anti-β1-integrin at the expected 130 kDa. The right panels (B) show the effects of using anti-β1-integrin as the immunoprecipitating antibody. A dense band, present at 130 kDa, stained with anti-β1-integrin, and a fainter band, present at 60-65 kDa, stained with anti CD46. The top two panels show CD46, while the bottom two panels show β1 integrin. The markers for the top two panels represent 120 and 160 kDa, while the markers for the bottom two panels represent 10, 60, and 70 kDa.

FIG. 8 depicts a cell adhesion assay. (A) The top panel shows inhibition of RPE attachment by anti-CD46 was dose-dependent. RPE cells were incubated with various concentrations of anti-CD46 antibodies, before seeding onto the basal lamina layer of human Bruch's membrane. Anti-CD46 antibody inhibited approximately 40-50% of cell attachment, with half-maximum inhibition at a concentration of 1 μg/mL. The X axis represents concentration of anti-CD46, in μg/mL; the Y axis represents % RPE attachment. (B) The bottom panel shows that anti-β1-integrin (10 μg/mL) produced inhibition similar to that of anti-CD46 (10 μg/in L). Control RPE cells were incubated with a non-immune IgG (mouse IgG1κ MOPC21 monoclonal antibody) to the RPE before seeding onto Bruch's membrane. Incubation with both antibodies did not cause any additional effect. The left axis represents % RPE attachment. The bars along the bottom axis represent, from left to right: anti-CD46+anti-β1-integrin, anti-β1-integrin, anti-CD46, and IgG.

### DETAILED DESCRIPTION OF THE INVENTION

As discussed below, the inventors have discovered a role for CD46, a complement regulatory protein, in retinal pigment epithelium (RPE) cell adhesion. RPE was obtained from human donor eyes, and from human immortalized RPE cell lines (ARPE19). Immunohistochemistry and confocal microscopy were then used to immunolocalize CD46 and β1 integrin. Immunoprecipitation experiments were performed on RPE cell lysates, using antibodies to either CD46 or β1 integrin. Thereafter, a cell adhesion assay was used to determine the proportion of RPE cells that adhered to Bruch's membrane explants from donor eyes.

The inventors have demonstrated that CD46, a complement regulatory protein, preferentially localized to the basolateral membrane surface in RPE from human donor eyes in situ, along with β1 integrin, and also preferentially localized in primary cultures of human RPE and in ARPE19 cell lines. Immunoprecipitation experiments of RPE lysates from the same sources demonstrated that CD46 co-precipitated with β1 integrin; in the reverse immunoprecipitation protocol, β1 integrin co-precipitated with CD46, indicating a physical relationship between the two proteins. Functional blocking of RPE adhesion with antibodies to CD46 confirmed that anti-CD46 reduces RPE cell adhesion; this is similar to the effect that has been shown with anti-β1-integrin antibodies (Ho and Del Priore, Reattachment of cultured human retinal pigment epithelium to extracellular matrix and human Bruch's membrane. Invest. Ophthalmol. Vis. Sci., 38:1110-18, 1997). Incubation with antibodies to both CD46 and β1 integrin inhibited RPE adhesion to the same extent as either antibody alone, suggesting that both antibodies affect the same site.

The inventors' findings indicate that CD46, a complement regulatory protein that protects host cells from autologous complement attack, has a functional interaction with β1 integrin in the eye, and that this interaction is related to adhesion of RPE to its basement membrane and to Bruch's membrane. Although it has been shown that CD46 associates directly with multiple β1 integrins in non-ocular tissue (Lozahic et al., CD46 (membrane cofactor protein) associates with multiple beta 1 integrins and tetraspans. Eur. J. Immunol., 30:900-07, 2000), this is the first report in ocular tissue of a functional interaction between a complement regulatory protein and a β1 integrin. This finding may be significant, not only in adhesive mechanisms in the retina, but in a constellation of functional interactions associated with integrin signaling pathways.

CD46, or membrane cofactor protein (MCP), is expressed on all nucleated human cells. It acts to protect the host cell against autologous complement attack, by degrading C3b (Liszewski and Atkinson, Membrane cofactor protein. Curr. Top. Microbiol. Immunol., 178:45-60, 1992; Seya et al., Human membrane cofactor protein (MCP, CD46): multiple isoforms and functions. Int. J. Biochem. Cell Biol., 31:1255-60, 1999). CD46 is also present on the basolateral surface of polarized epithelial cells (Maisner et al., Two different cytoplasmic tails direct isoforms of the membrane cofactor protein (CD46) to the basolateral surface of Madin-Darby canine kidney cells. J. Biol. Chem., 271:18853-58, 1996; Maisner et al., Membrane cofactor protein (CD46) is a basolateral protein that is not endocytosed: importance of the tetrapeptide FTSL at the carboxyl terminus. J. Biol. Chem., 272:20793-99, 1997; Teuchert et al., Importance of the carboxy-terminal FTSL motif of membrane cofactor protein for basolateral sorting and endocytosis: positive and negative modulation by signals inside and outside the cytoplasmic tail. J. Biol. Chem., 274:19979-84, 1999; Ludford-Menting et al., A functional interaction between CD46 and DLG4: a role for DLG4 in epithelial polarization. J. Biol. Chem., 277:4477-84, 2002). This localization is similar to that shown by the inventors in the current RPE study. Further studies of the basolateral targeting mechanism have demonstrated that there is a functional interaction between CD46 and DLG4, a member of the guanylate kinase family, and that the polarized expression of CD46 in epithelial cells requires the DLG4-binding domain (Ludford-Menting et al., A functional interaction between CD46 and DLG4: a role for DLG4 in epithelial polarization. J. Biol. Chem., 277:4477-84, 2002).

One of the proteins belonging to the DLG4 family may also have a functional interaction with CD46 in RPE cells. DLG4 is one of a family of four human proteins that share a single homologue with a tumor-suppressor protein, discharge (DLG), from Drosophila (Woods and Bryant, The discs-large tumor suppressor gene of Drosophila encodes a guanylate kinase homolog localized at septate junctions. Cell, 66:451-64, 1991; Fujita and Kurachi, SAP family proteins. Biochem. Biophys. Res. Commun., 269:16, 2000; Dimitratos et al., Signaling pathways are focused at specialized regions of the plasma membrane by scaffolding proteins of the MAGUK family. Bioessays, 21:912-21, 1999). DLG4 is also called “postsynaptic density” (PSD) or “synapse-associated protein” (SAP), because of its localization in the postsynaptic density region of neurons.

DLG4 proteins halve multiple protein-protein interaction motifs, including three PDZ domains. The PDZ domain is a recognition domain that derives its name from the three proteins first characterized as having such domains: PSD from neurons, DLG from Drosophila, and ZO1 from tight junctions. DLG4 proteins, which are polarized, interact with other PDZ-domain-containing proteins, membrane receptors, cell adhesion molecules, and the cytoskeleton, to regulate epithelial cell polarization and assemble signaling cascades (Kim, S. K., Polarized signaling: basolateral receptor localization in epithelial cells by PDZ containing proteins. Curr. Opin. Cell Biol., 9:853-59, 1997; Fanning and Anderson, PDZ domains: fundamental building blocks in the organization of protein complexes at the plasma membrane. J. Chem. Invest., 103:767-72, 1999; Fanning and Anderson, Protein modules as organizers of membrane structure. Curr. Opin. Cell Biol., 11:432-39, 1999; Garner et al., PDZ domains in synapse assembly and signaling. Trends Cell Biol., 10:274-80, 2000).

In view of the foregoing, it is of interest that recent studies of rat RPE have shown that one of the PDZ-domain-containing proteins, SAP-97, localizes to the basolateral surface of RPE, and may have a direct interaction with ezrin (Bonilha and Rodriguez-Boulan, Polarity and developmental regulation of two PDZ proteins in the retinal pigment epithelium. Invest. Ophthalmol. Vis. Sci., 42:3274-82, 2001), an actin-binding protein associated with the morphogenesis of RPE apical microvilli and basal infoldings (Bonilha et al., Ezrin promotes morphogenesis of apical microvilli and basal infoldings in retinal pigment epithelium. J. Cell Biol., 147:15533-48, 1999). Ezrin belongs to the ERM family of proteins, which defines three highly-homologous proteins (ezrin, radixin, moesin) that constitute a group of plasma membrane/cytoskeleton linkers that regulate cell adhesion and morphogenesis of the actin rich cell cortex (Mangeat et al., ERM proteins in cell adhesion and membrane dynamics. Trends Cell Biol., 9:187-92, 1999).

Cell adhesion to the extracellular matrix (ECM) is a crucial regulator of cell behavior. Large protein complexes of signaling proteins and cytoskeleton are assembled into functional units at the sites of integrin/matrix adhesion (Schwartz, M. A., Integrin signaling revisited. Trends Cell Biol., 11:466-70, 2001). The inventors previously showed that the β1 subunit of integrin partially mediates the adherence of human RPE cells to RPE-derived extracellular matrix and the basal lamina layer of human Bruch's membrane (Ho and Del Priore, Reattachment of cultured human retinal pigment epithelium to extracellular matrix and human Bruch's membrane. Invest. Ophthalmol. Vis. Sci., 38:1110-18, 1997). More recently, immunoprecipitation experiments in carcinoma-derived cell lines showed that CD46 associates with multiple β1 integrins (Lozahic et al., CD46 (membrane cofactor protein) associates with multiple beta 1 integrins and tetraspans. Eur. J. Immunol., 30:900-07, 2000), and associates indirectly with a superfamily of surface molecules, the tetraspanins (Maecker et al., The tetraspanin superfamily: molecular facilitators. FASEB J., 11:428-42, 1997).

Tetraspanins are known to associate with a subset of β1 integrins (Rubinstein et al., CD9 antigen is an accessory subunit of the VLA integrin complexes. Eur. J. Immunol., 24:3005-13, 1994; Rubinstein et al., CD9, CD63, CD81, and CD82 are components of a surface tetraspan network connected to HLA-DR and VLA integrins. Eur. J. Immunol., 26:2657-65, 1996: Berditchevski et al., Characterization of novel complexes on the cell surface between integrins and proteins with four transmembrane domains (TM4 proteins). Mol. Biol. Cell, 7:193-207, 1996; Radford et al., CD63 associates with transmembrane 4 superfamily members, CD9 and CD81, and with beta 1 integrins in human melanoma. Biochem. Biophys. Res. Commun. 222:13-18, 1996). It is also known that tetraspanins form a web with common functions related to migration, proliferation, intracellular signaling, and adhesion (Ikeyama et at., Suppression of cell motility and metastasis by transfection with human motility-related protein (MRP-1/CD9) DNA. J. Exp. Med., 177:1231-37, 1993; Radford et al., Regulation of tumor cell motility and migration by CD63 in a human melanoma cell line. J. Immunol., 158:3353-58, 1997; Dong et (al., Down-regulation of the KA11 metastasis suppressor gene during the progression of human prostatic cancer infrequently involves gene mutation or allelic loss. Cancer Res., 56:4387-90, 1996; Lagaudriere-Gesbert et al., Functional analysis of foul tetraspans, CD9, CD53, CD81, and CD82, suggests a common role in costimulation, cell adhesion, and migration: only CD9 upregulates HB-EGF activity. Cell Immunol., 182:105-12, 1997; Horvath et al., CD19 is linked to the integrin-associated tetraspans CD9, CD81, and CD82. J. Biol. Chem., 273:30537-43, 1998). CD46 is a newly discovered component of this web (Lozahic et al., CD46 (membrane cofactor protein) associates with multiple beta 1 integrins and tetraspans. Eur. J. Immunol., 30:900-07, 2000).

In connection with RPE cells, the above-described protein associations are relevant because they are believed to form the functional units underlying normal adhesion mechanisms, and, therefore, are involved in maintaining a healthy RPE phenotype that is not proliferative or migratory. When there is disease, the functional units become disrupted, the RPE loses attachment to Bruch's membrane, and RPE cells may break away from the monolayer and undergo apoptosis (Tezel et al., Reattachment to a substrate prevents apoptosis of human retinal pigment epithelium. Graefes Arch. Clin. Exp. Ophthalmol., 235:41-47, 1997). Therefore, the loss of RPE cells—one of the first signs of ARMD (Del Priore et al. in New Developments in the Treatment of Age-Related Macular Degeneration, Capone et al., eds. (Verona, IT: Progei Editori, 1997) pp. 14551) may be preceded by the loss of RPE attachment to Bruch's membrane, resulting from a dysfunctional CD46/β1 integrin complex.

A recent study, in which flat-mount preparations of human cadaveric eyes were stained with the TUNEL technique, provides direct evidence that human RPE undergoes age-related apoptosis in situ, with apoptotic human RPE confined mainly to the macula of older human eyes (Del Priore et al., Age-related changes in human RPE cell density and apoptosis proportion in situ. Invest. Ophthalmol. Vis. Sci., 43:3312-18, 2002). Another similar report suggests that human RPE die by apoptosis around the edges of geographic atrophy (Dunaief et al., The role of apoptosis in age-related macular degeneration. Arch. Ophthalmol., 120:1435-42, 2002). Secondary atrophy of the underlying choriocapillaris and overlying photoreceptors would then follow, signalling the clinical recognition of ARMD.

In view of the foregoing, the inventors expect that their discovery, as described herein may have applications in procedures which require inhibition of cell attachment, proliferation, and/or migration. By way of example, it is well known that prosthetic implants cause tissue interactions between hosts and artificial implants. For example, fibrous encapsulation of subretinal prostheses is a major problem. It is expected that antibodies to CD46 and/or integrin will alleviate this problem. The inventors' discovery may halve application to other types of procedures that require implantation of a prosthetic device. A prosthetic device, such as a coronary arterial stent, could be coated with antibodies to CD46 and/or integrin. These antibodies would serve to inhibit cell attachment to the device, and to inhibit cell proliferation, which is a major problem. Antiproliferative drugs are currently used on such stents to inhibit the proliferation process. Additionally, antibodies to CD46 and/or integrin could be used to inhibit the cell adhesion required for neoplastic growth in some cancers.

Accordingly, the present invention provides a medical device for use in inhibiting adhesion of cells (e.g., epithelial cells), wherein the medical device has a coating comprising at least one inhibitor of CD46. The medical device may be particularly useful for preventing, treating, or inhibiting the occurrence of conditions associated with cell adhesion, migration, and proliferation, including, without limitation, fibrous encapsulation of retinal and subretinal prostheses, restenosis, restenosis after angioplasty and/or stent implantation, and accelerated arteriopathy after cardiac transplantation.

In the method of the present invention, CD46 may be inhibited by disabling, disrupting, or inactivating the function and/or activity of CD46, and/or by diminishing the amount, level, and/or expression of CD46 in a cell, tissue, or subject. Furthermore, CD46 function/activity or level/expression may be inhibited by targeting CD46 directly, and/or by targeting CD46 in an indirect manner (e.g., by directly or indirectly causing, inducing, or stimulating the down-regulation of CD46 activity or expression within a cell or tissue). Preferably, the function/activity or level/expression of CD46 in the cell, tissue, or subject is inhibited by at least 10% in the method of the present invention. More preferably, CD46 in the cell, tissue, or subject is inhibited by at least 20%.

CD46, or membrane cofactor protein (MCP), is a ubiquitous transmembrane glycoprotein expressed on most human cells, apart from erythrocytes. CD46 is a complement inhibitor protein which plays a major role in regulating the immune response and in protecting normal autologous tissues from complement-mediated destruction. Specifically, CD46 binds to the complement immune component, C3b, promoting the activity of proteases that cleave C3b into inactive fragments, and thereby preventing the cell from autologous attack. CD46 serves as a receptor for measles virus and other human pathogens, and is highly expressed at blood-brain barriers. Importantly, CD46 is localized on the basolateral membrane of epithelial cells, including retinal pigment epithelial (RPE) cells.

As used herein, “a CD46 inhibitor” or “an inhibitor of CD46” shall include a protein, polypeptide, peptide, nucleic acid (including DNA, RNA, and an antisense oligonucleotide), antibody (monoclonal or polyclonal), Fab fragment, F(ab′)₂ fragment, molecule, compound, antibiotic, drug, and any combination thereof, that inhibits CD46. A Fab fragment is a univalent antigen-binding fragment of an antibody, which is produced by papain digestion. A F(ab′)₂ fragment is a divalent antigen-binding fragment of an antibody, which is produced by pepsin digestion. The CD46 inhibitor may be an agent reactive with CD46 (i.e., an agent that has affinity for, binds to, or is directed against CD46) and/or it may directly or indirectly inhibit or down-regulate CD46 expression. By way of example, the CD46 inhibitor of the present invention may be a CD46 transgene, comprising the CD46 gene and an inducible promoter, in the absence of a suitable inducer. In a cell containing such a transgene, CD46 expression would be sustained in the presence of a suitable inducing agent; however, CD46 expression would be shut down once the supply of inducer was depleted. Thus, a CD46 transgene, comprising the CD46 gene and an inducible promoter, would, in the absence of a suitable inducer, effectively bring about a decrease in the amount or level of CD46 in the cell, thereby functioning as a CD46 inhibitor.

The CD46 inhibitor of the present invention also may be an interfering RNA, or RNAi, including CD46 small interfering RNA (siRNA). As used herein, “RNAi” refers to a double-stranded RNA (dsRNA) duplex of any length, with or without single-strand overhangs, wherein at least one strand, putatively the antisense strand, is homologous to the target mRNA to be degraded. As further used herein, a “double-stranded RNA” molecule includes any RNA molecule, fragment, or segment containing two strands forming an RNA duplex, notwithstanding the presence of single-stranded overhangs of unpaired nucleotides. Additionally, as used herein, a double-stranded RNA molecule includes single-stranded RNA molecules forming functional stem-loop structures, such that they thereby form the structural equivalent of an RNA duplex with single-strand overhangs. The double-stranded RNA molecule of the present invention may be very large, comprising thousands of nucleotides; preferably, however, it is small, in the range of 2125 nucleotides. In a preferred embodiment, the RNAi of the present invention comprises a double-stranded RNA duplex of at least 19 nucleotides.

In one embodiment of the present invention. RNAi is produced in vivo by an expression vector containing a gene-silencing cassette coding for RNAi (see, e.g., U.S. Pat. No. 6,278,039, C. elegans deletion mutants; U.S. Patent Application No. 2002/0006664, Arrayed transfection method and uses related thereto; WO 99/32619, Genetic inhibition by double-stranded RNA; WO 01/29058, RNA interference pathway genes as tools for targeted genetic interference; WO 01/68836, Methods and compositions for RNA interference; and WO 01/96584. Materials and methods for the control of nematodes). In another embodiment of the present invention, RNAi is produced in vitro, synthetically or recombinantly, and is then transferred into the microorganism using standard molecular-biology techniques. Methods of making and transferring RNAi are well known in the art. See, e.g., Ashrafi et al., Genome-wide RNAi analysis of Caenorhabditis elegans fat regulatory genes. Nature, 421:268-72,2003; Cottrell et al., Silence of the strands: RNA interference in eukaryotic pathogens. Trends Microbiol., 11:37-43, 2003; Nikolaev et al., Parc. A Cytoplasmic Anchor for p53. Cell, 112:29-40, 2003; Wilda et al., Killing of leukemic cells with a BCR/ABL fusion gene RNA interference (RNAi). Oncogene, 21:5716-24,2002; Escobar et al., RNAi mediated oncogene silencing confers resistance to crown gall tumorigenesis. Proc. Natl. Acad. Sci. USA, 98:13437-42, 2001; and Billy et al., Specific interference with gene expression induced by long, double-stranded RNA in mouse embryonal teratocarcinoma cell lines. Proc. Natl. Acad. Sci. USA, 98:14428-33, 2001.

Additionally, the CD46 inhibitor of the present invention may be an oligonucleotide antisense to CD46. Oligonucleotides antisense to CD46 may be designed based on the nucleotide sequence of CD46, which is readily available. For example, a partial sequence of the CD46 nucleotide sequence (generally, 1820 base pairs), or a variation sequence thereof, may be selected for the design of an antisense oligonucleotide. This portion of the CD46 nucleotide sequence may be within the 5′ domain. A nucleotide sequence complementary to the selected partial sequence of the CD46 gene, or the selected variation sequence, then may be chemically synthesized using one of a variety of techniques known to those skilled in the art, including, without limitation, automated synthesis of oligonucleotides having sequences which correspond to a partial sequence of the CD46 nucleotide sequence, or a variation sequence thereof, using commercially-available oligonucleotide synthesizers, such as the Applied Biosystems Model 392 DNA/RNA synthesizer.

Once the desired antisense oligonucleotide has been prepared, its ability to inhibit CD46 then may be assayed. For example, the oligonucleotide antisense to CD46 may be contacted with retinal pigment epithelial cells, or lens epithelial cells, and the levels of CD46 activity or expression in the cells may be determined using standard techniques, such as Western-blot analysis and immunostaining. Alternatively, the antisense oligonucleotide may be delivered to retinal pigment epithelial cells, or lens epithelial cells, using a liposome vehicle, and the levels of CD46 activity or expression in the cells may be determined using standard techniques, such as Western-blot analysis. Where the level of CD46 activity or expression in the cells is reduced in the presence of the designed antisense oligonucleotide, it may be concluded that the oligonucleotide could be a useful CD46 inhibitor.

It is within the confines of the present invention that oligonucleotide antisense to CD46 may be linked to another agent, such as a drug or a ribozyme, in order to increase the effectiveness of treatments using CD46 inhibition, to increase the efficacy of targeting, and/or to increase the efficacy of degradation of CD46 RNA. Moreover, oligonucleotide antisense to CD46 may be prepared using modified bases (e.g., a phosphorothioate), to make the oligonucleotide more stable and better able to withstand degradation.

In a preferred embodiment, the CD46 inhibitor of the present invention is an antibody that binds to CD46 (e.g., is specific for CD46). As used herein, the anti-CD46 antibody may be polyclonal or monoclonal. In addition, the anti-CD46 antibody may be produced by techniques well known to those skilled in the art. For example, polyclonal antibody may be produced by immunizing a mouse, rabbit, or rat with purified CD46, or with a short peptide sequence thereof. Monoclonal antibody then may be produced by removing the spleen from the immunized mouse, and fusing the spleen cells with myeloma cells to form a hybridoma which, when grown in culture, will produce a monoclonal antibody.

The CD46 inhibitor of the present invention also may be a dominant-negative form of the protein, including a dominant-negative form of CD46 expressed on an inducible promoter. Additional CD46 inhibitors may be identified using screening procedures well known in the art and/or described herein. It is to be understood that a number of compounds or agents that are not listed herein also inhibit CD46. Accordingly, the description of exemplary CD46 inhibitors set forth herein is not limited thereto.

The present invention contemplates the use of proteins and protein analogues generated by synthesis of polypeptides in vitro, e.g., by chemical means or in vitro translation of mRNA. For example, CD46 and inhibitors thereof may be synthesized by methods commonly known to one skilled in the art (Modern Techniques of Peptide and Amino Acid Analysis (New York: John Wiley & Sons, 1981); Bodansky, M., Principles of Peptide Synthesis (New York: Springer-Verlag New York, Inc., 1984). Examples of methods that may be employed in the synthesis of the amino acid sequences, and analogues of these sequences, include, but are not limited to, solid-phase peptide synthesis, solution-method peptide synthesis, and synthesis using any of the commercially-available peptide synthesizers. The amino acid sequences of the present invention may contain coupling agents and protecting groups, which are used in the synthesis of protein sequences, and which are well known to one of skill in the art.

In the medical device of the present invention, the CD46 inhibitor may be provided in an amount effective to inhibit adhesion of cells in a subject. The subject may be any animal, including amphibians, birds, fish, mammals, and marsupials, but is preferably a mammal (e.g., a human; a domestic animal, such as a cat, dog, monkey, mouse, and rat; or a commercial animal, such as a cow or pig). In a preferred embodiment, the subject is a human. An effective amount of a CD46 inhibitor compound generally refers to an amount and/or concentration of inhibitor necessary to achieve a desired result in this case, adhesion of cells, particularly adhesion of cells to the medical device. Accordingly, it is also understood that the effective amount of CD46 inhibitor on the medical device may vary. For instance, the effective amount may vary depending upon the desired amount or degree of cell-adhesion inhibition, the subject's weight, severity of the subject's condition, etc.

The medical device of the present invention may be used to inhibit adhesion of cells in a subject by introducing the medical device into the subject at a site susceptible to adhesion of cells. It is to be understood that the present invention may be used to limit cell adhesion in a variety of tissues, including ocular tissue (e.g., lens, retina) and venous and arterial blood vessels. It is also understood that the medical device of the present invention may be designed for use in various types of medical procedures. The medical device is preferably introduced to the subject intravascularly; however, the device may also be introduced into the subject via open surgical intervention.

In accordance with the present invention, a medical device coated with a CD46 inhibitor will be generally capable of inhibiting adhesion of cells in a subject, particularly epithelial cells in the subject (e.g., lens or RPE cells in the subject's eye, or smooth muscle cells in the subject's vasculature). Specifically, the medical device will be capable of inhibiting the adhesion of cells to the device resulting from use of the device in a subject. Because cells generally need to adhere to a surface (e.g., an extracellular matrix) in order to migrate or proliferate, inhibition of cell adhesion to the device may serve further to inhibit any related proliferation and/or migration of cells. Thus, it is within the confines of the present invention that the medical device may also be used to inhibit the processes of cellular proliferation and/or migration.

In one embodiment of the present invention, the medical device is a prosthetic device. By way of example, the prosthetic device may be an intraocular lens (e.g., an artificial substitute for a lens) or a retinal or subretinal prosthesis (e.g., an artificial substitute for a retina, used for functional and/or cosmetic reasons, which may be inserted between the sensory retina and the retinal pigment epithelium or between the retinal pigment epithelium and the choroid). Such devices, when coated with one or more CD46 inhibitors, may inhibit fibrous encapsulation of the prostheses. The prosthetic device of the present invention also may be a coated stent for implantation in a subject's blood vessel, for use in maintaining the patency of the vessel. The stent may be implanted in connection with an angioplasty procedure or in other instances or procedures that may trigger adhesion of smooth muscle cells including, without limitation, vascular injury, graft implantation or transplantation, and cardiac transplantation.

In another embodiment, the medical device of the present invention is a catheter, such as an angioplasty balloon catheter, which, when coated with at least one CD46 inhibitor, may inhibit adhesion of smooth muscle cells (SMCs) during the initial injury caused by opening the occluded blood vessel therewith. Catheters coated with CD46 inhibitors may also aid in preventing or treating SMC adhesion that results from injury to blood vessels arising from navigation of the catheter to a site in the subject where an intravascular intervention procedure will occur. Cell adhesion may further be inhibited with a combination of coated medical devices, including a coated stent in combination with a coated balloon catheter. In such an instance, the combination would provide CD46 inhibitor compounds at all stages of the angioplasty and stenting procedures.

The medical device of the present invention may be coated with non thrombogenic or thrombolytic agents that inhibit the formation of, or that break up, a thrombus. An example of such an agent is heparin. The medical device of the present invention also may be manufactured from a variety and/or a combination of biocompatible and non-biocompatible materials, including, without limitation, polyester, Gortex, polytetrafluoroethyline (PTFE), polyethelene, polypropylene, polyurethane, silicon, steel, stainless steel, titanium, Nitinol or other shape-memory alloys, copper, silver, gold, platinum, Kevlar fiber, and carbon fiber. Where non-biocompatible materials may come into contact with a subject's anatomy, the components made from the non-biocompatible materials may be covered or coated with a biocompatible material.

Additionally, the medical device of the present invention may be coated using a variety of techniques, including dipping, spraying, etc. In one embodiment, the medical device, particularly a stent, is coated with at least one biodegradable carrier, such as a degradable or erodeable polymer, which includes therein an effective amount of the CD46 inhibitor. The biodegradable carrier degrades over time, thereby allowing the CD46 inhibitor (or other compound or agent therein) to elute from the medical device over time. The term “elute” is used herein to denote the release or separation of a compound or agent from the medical device, and is not limited to any particular mechanism unless otherwise noted. The medical device may be coated with the biodegradable carrier in various thicknesses. Generally, the greater the thickness of the coating, the longer it will take for the inhibitor, compound, or agent therein to elute from the medical device. The preferred duration of therapy would range from 7 days to 2 months.

In another embodiment of the present invention, the medical device is coated with a biodegradable carrier in layers, with each coating or layer providing a different or additional active ingredient (e.g., another CD46 inhibitor, a non-thrombogenic agent, etc.), thereby providing timed release of the active ingredient. For example, the medical device may be coated with a first layer that consists of a biodegradable carrier, a CD46 inhibitor, and a non-thrombogenic agent, and a second layer that consists of a biodegradable carrier and the same or another CD46 inhibitor. In this instance, the non-thrombogenic agent may be eluted for a limited time (e.g., during degradation of the first layer), or in a timed-release manner. Additionally, this embodiment would permit elution of different types of active ingredients (e.g., CD46 inhibitors) at different times (e.g., a first CD46 inhibitor may be eluted during degradation of the first layer, and a second CD46 inhibitor may be eluted during degradation of the second layer), in a timed-release manner. In a preferred embodiment, the present invention provides a stent for implantation in a blood vessel, wherein the stent has a coating comprising a biodegradable carrier that degrades over time and a CD46 inhibitor, and wherein the CD46 inhibitor is an anti-CD46 antibody.

In a further embodiment of the present invention, different sides of the medical device may be coated in single or multiple layers (e.g., a plurality of coatings) with biodegradable carriers which include therein different active ingredients, thereby permitting staged release of the active ingredients. For instance, the exterior side of a medical device (e.g., the portion which, when implanted in a subject, contacts the subject's vasculature), may be coated with a biodegradable carrier which includes a CD46 inhibitor; the opposite side of the device, which is exposed to a subject's blood, may be coated with a non-thrombogenic agent or a biodegradable carrier containing a non-thrombogenic agent. In another embodiment, the medical device includes therein structures, such as pores or other reservoir systems, which are capable of holding the CD46 inhibitor. In this instance, a suitable release mechanism, such as a membrane, may be used to release the CD46 inhibitor from the medical device.

The medical device of the present invention may also have a coating comprising, both an inhibitor of CD46 and an inhibitor of β integrin. The integrins form a superfamily of transmembrane heterodimers, each composed of an alpha and beta subunit. In general, integrins are subclassified into β1, β2 and β3 subtypes. β1 integrin is a cell-surface protein complex which functions in both cell-substrate and cell-to-cell adhesion. In a preferred embodiment of the present invention, the β integral is β1 integrin. As used herein, “an inhibitor of β integrin” or “a β integrin inhibitor” shall include a protein, polypeptide, peptide, nucleic acid (including DNA, RNA, and an antisense oligonucleotide), antibody (monoclonal or polyclonal), Fab fragment, F(ab)₂ fragment, molecule, compound, antibiotic, drug, and any combination(s) thereof that inhibits β integrin. In a preferred embodiment of the present invention, the β integrin inhibitor is an antibody that binds to β integrin (e.g., is specific for β integrin).

In view of the foregoing, the present invention further provides a use of a medical device in a method for inhibiting adhesion of cells, wherein the medical device has a coating comprising a CD46 inhibitor. In one embodiment, the coating of the medical device further comprises an inhibitor of β integrin. Additionally, the present invention provides a use of a medical device in a method for inhibiting adhesion of cells, wherein the medical device has a coating comprising a biodegradable carrier that degrades over time and a CD46 inhibitor. In one embodiment, the coating of the medical device further comprises an inhibitor of β integrin.

The present invention further provides a method for inhibiting cell-substrate adhesion, or inhibiting adhesion of a cell to a substrate. As used herein, the term “substrate” includes any surface upon which a cell may attach or to which a cell may adhere. Examples of substrates include, without limitation, other cells (either in culture or in a subject), an extracellular matrix (e.g., Bruch's membrane or the basement membrane to which RPE cells attach), a medical device (e.g., an intraocular lens, a retinal or subretinal prosthesis, a catheter, or a stent). As further used herein, the term “inhibiting adhesion” includes blocking, decreasing, inhibiting, limiting, or preventing the adhesion, attachment, or physical association of a cell to a surface, such as a substrate or another cell. Inhibition of cell-surface adhesion may also result in inhibition of cell proliferation (e.g., cell growth and/or cell division) and inhibition of cell migration, which often depend upon attachment of cells to a surface. Inhibition of cell adhesion, and inhibition of cell proliferation and migration, may be detected by known procedures, including any of the methods, molecular procedures, and assays disclosed herein.

The cell of the present invention may be any cell, but is preferably a CD46 expressing cell. As further used herein, the term “CD46-expressing cell” refers to a cell that expresses CD46—whether naturally or as a result of engineering. The terms “expressing” and “expression,” as used herein, mean the transcription of a gene into at least one mRNA transcript, or the translation of at least one mRNA into a protein. In one embodiment of the present invention, the cell is all epithelial cell (e.g., a lens or RPE cell, or a vascular smooth muscle cell). In another embodiment, the cell of the present invention is a neoplastic cell. Neoplastic cells are cells of a neoplasia. As used herein, the term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Neoplasms include benign tumors and malignant tumors that are either invasive or non invasive. Malignant neoplasms are distinguished from benign in that the former show a greater degree of anaplasia, or loss of differentiation and orientation of cells, and have the properties of invasion and metastasis. Thus, neoplasia includes “cancer”, which herein refers to a proliferation of tumor cells having the unique trait of loss of normal controls, resulting in unregulated growth, lack of differentiation, local tissue invasion, and/or metastasis.

In accordance with the present invention, adhesion of a cell to the substrate may be inhibited by inhibiting CD46 in the cell. Unless otherwise indicated, “CD46” includes both a CD46 protein and a “CD46 analogue”. As used herein, a “CD46 analogue” is a functional variant of the CD46 protein, having CD46 biological activity, that has 60% or greater (preferably, 70% or greater) amino-acid-sequence homology with the CD46 protein. As further used herein, the term “CD46 biological activity” refers to the activity of a protein or peptide that demonstrates an ability to associate physically with β1 integrin (i.e., binding of approximately two fold, or, more preferably, approximately five fold, above the background binding of a negative control), and/or an ability to facilitate cell adhesion, as described herein. CD46 and CD46 analogues may be produced synthetically or recombinantly, or may be isolated from native cells.

In the method of the present invention, CD46 may be inhibited in the cell by contacting the cell with an inhibitor of CD46. The CD46 inhibitor is provided in an amount that is effective to inhibit adhesion of a cell to a substrate. This amount may be readily determined by the skilled artisan, based upon known procedures and methods disclosed herein. Examples of CD46 inhibitors are described above. Preferably, the CD46 inhibitor is an anti-CD46 antibody.

In one embodiment, the method of the present invention further comprises the step of inhibiting β integrin (e.g., β integrin) in the cell. β integrin may be inhibited in the cell by contacting the cell with an inhibitor of β integrin. The β integrin inhibitor is provided in an amount which, in combination with an effective amount of CD46 inhibitor, is effective to inhibit adhesion of a cell to a substrate. This amount may be readily determined by the skilled artisan, based upon known procedures and methods disclosed herein. Examples of β integrin inhibitors are described above. Preferably, the β integrin inhibitor is an anti-βintegrin antibody.

In the method of the present invention, one or more cells may be contacted with effective amounts of CD46 and β integrin inhibitors either in vitro, or in vivo in a subject. The inhibitors may be contacted with the cells by introducing the inhibitors into the cells. Where contacting is effected in vitro, the inhibitors may be added directly to the culture medium. Alternatively, the inhibitors may be contacted with cells ill vivo in a subject, by introducing the inhibitors into the subject (e.g., by introducing the inhibitors into cells of the subject) and/or by administering the inhibitors to the subject. The subject may be any of those described above. In a preferred embodiment, the subject is known, or believed, to be susceptible to a condition associated with adhesion and/or proliferation of cells.

Where the inhibitors are contacted with cells ill vivo, the subject is preferably a human. The cells may be contained in any tissue of a subject (e.g., lens epithelium, retinal pigment epithelium, smooth muscle epithelium, etc.), and may be detected in tissue of the subject by standard detection methods readily determined from the known art. Examples of such methods include, without limitation, immunological techniques (e.g., immuno histochemical staining), fluorescence imaging techniques, and microscopic techniques.

The inhibitors of the present invention may be contacted with cells, either ill vitro or in vivo in a subject, by known techniques used for the introduction and administration of proteins, nucleic acids, and other drugs. Examples of methods for contacting the cells with (i.e., treating the cells with) a CD46 or β integrin inhibitor (in molecule, protein, or nucleic acid form) include, without limitation, absorption, electroporation, immersion, injection, introduction, liposome delivery, transfection, transfusion, vectors, and other drug-delivery vehicles and methods. When target cells are localized to a particular portion of a subject, it may be desirable to introduce the inhibitors directly to the cells, by injection or by some other means (e.g., by introducing the inhibitors into the blood or another body fluid).

Where the inhibitor is a protein or other molecule, it may be introduced into a cell directly, in accordance with conventional techniques and methods disclosed herein. Additionally, a protein inhibitor may be introduced into a cell indirectly, by introducing into the cell a nucleic acid encoding the inhibitor, in a manner permitting expression of the protein inhibitor. The inhibitor may be introduced into cells, in vitro or in vivo, using conventional procedures known in the art, including, without limitation, electroporation, DEAE Dextran transfection, calcium phosphate transfection, monocationic liposome fusion, polycationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer, or any combination thereof. Recombinant viral vectors suitable for gene therapy include, but are not limited to, vectors derived from the genomes of such viruses as retrovirus, HSV, adenovirus, adeno-associated virus, Semiliki Forest virus, cytomegalovirus, lentivirus, and vaccinia virus. The amount of nucleic acid to be used is an amount sufficient to express an amount of protein effective to inhibit adhesion of a cell to a substrate. This amount may be readily determined by the skilled artisan.

It is also within the confines of the present invention that a nucleic acid encoding a protein inhibitor may be introduced into suitable cells in vitro, using conventional procedures, to achieve expression of the protein inhibitor in the cells. Cells expressing the protein inhibitor then may be introduced into a subject to inhibit adhesion of cells in vivo.

In accordance with the method of the present invention, CD46 and β integrin inhibitors may be administered to a human or animal subject by known procedures including, without limitation, oral administration, parental administration (e.g., epifascial intracapsular, intracutaneous, intradermal, intramuscular, intraorbital, intraperitoneal, Intraspinal, intrasternal, intravascular, intravenous, parenchlymatous, or subcutaneous administration), transdermal administration, administration by osmotic pump, and implantation, introduction, or insertion of an inhibitor-coated medical device. For oral administration, the CD46 or β integrin inhibitor may be presented as capsules, tablets, powders, granules, or as a suspension. The inhibitor may be formulated with conventional additives, such as lactose, mannitol, cornstarch, or potato starch. The formulation may also be presented with binders, such as crystalline cellulose, cellulose derivatives, acacia, cornstarch, or gelatins. Additionally, the formulation may be presented with disintegrators, such as cornstarch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration, the CD46 or β integrin inhibitor may be combined with a sterile aqueous solution, which is preferably isotonic in relation to the blood of the subject. Such a formulation may be prepared by dissolving the inhibitor in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampules or vials. The formulation may be delivered by any mode of injection, including any of those described above.

For transdermal administration, the CD46 or β integrin inhibitor may be combined with skin-penetration enhancers, such as propylene glycol, polyethylene glycol, isopropanol, ethanol, oleic acid, N-methylpyrrolidone, and the like, which increase the permeability of the skin to the inhibitor, thereby allowing it to penetrate through the skin and into the bloodstream. The inhibitor may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The inhibitor may be administered transdermally, at or near the site on the subject where the cell adhesion is localized or is expected to arise. Alternatively, the inhibitor may be administered transdermally at a site other than the affected area, in order to achieve systemic administration. Preferably, the inhibitor of the present invention is administered locally.

The CD46 or β integrin inhibitor may also be released or delivered from an osmotic mini-pump or other timed-release device. The release rate from an elementary osmotic multi-pump may be modulated with a microporous, fast-response gel disposed in the release orifice. An osmotic mini-pump would be useful for controlling release, or targeting delivery, of the CD46 or β integrin inhibitor.

Additionally, the CD46 or β integrin inhibitor may be administered to a subject via a medical device (e.g., an intraocular lens, a retinal or subretinal prosthesis, or a stent) coated therewith, as described above. Such a device may be inserted, introduced, or implanted into a subject (e.g., in the subject's eye or in the subject's vasculature), at or near the site on the subject where the cell adhesion is localized or is expected to arise, thereby allowing the CD46 or β integrin inhibitor to elute from the device into the surrounding tissue. As described herein, the medical device may be constructed such that the CD46 inhibitor and/or another active ingredient is eluted from the device in a staged-release or timed-release manner. In one embodiment of the present invention, the medical device is implanted into a subject's vasculature in connection with a balloon angioplasty procedure. In another embodiment, local therapy is achieved with nanospheres impregnated with the CD46 or β integrin inhibitor (Chorny et al., Study of the drug release mechanism from tyrphostin AG 1295-loaded nanospheres by in situ and external sink methods. J. Controlled Release, 83:401-14, 2002).

It is also within the confines of the present invention that the CD46 or β integrin inhibitor may be further associated with a pharmaceutically-acceptable carrier, thereby comprising a pharmaceutical composition. Accordingly, the present invention further provides a pharmaceutical composition, comprising a CD46 inhibitor and a pharmaceutically acceptable carrier or a β integrin inhibitor and a pharmaceutically-acceptable carrier. The pharmaceutical carrier must be “acceptable” in the sense of being compatible with the other ingredients of the composition, and not deleterious to the recipient thereof. The pharmaceutically-acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include, without limitation, carboxymethyl cellulose, crystalline cellulose, glycerin, gum arabic, lactose, magnesium stearate, methyl cellulose, powders, saline, sodium alginate, sucrose, starch, talc, and water.

The pharmaceutical composition of the present invention may be prepared by methods well-known in the pharmaceutical arts. For example, the composition may be brought into association with a carrier or diluent, as a suspension or solution. Optionally, one or more accessory ingredients (e.g., buffers, flavoring agents, surface active agents, and the like) also may be added. The choice of carrier will depend upon the route of administration of the composition. Formulations of the composition may be conveniently presented in unit dosage, or in such dosage forms as aerosols, capsules, elixirs, emulsions, eye drops, injections, liquid drugs, pills, powders, granules, suppositories, suspensions, syrup, tablets, or troches, which can be administered by any of the modes of administration described above.

The present invention further provides a method for inhibiting cell-to-cell adhesion in a subject, i.e., for inhibiting adhesion between or among cells in a subject. Cell-to-cell adhesion is essential to many cellular activities, including the assembly and interconnection of various vertebrate systems, as well as maintenance of tissue integration, wound healing, morphogenic movements, cellular migrations, and metastasis.

The subject of the present invention may be any animal or human subject, as described above. Preferably, the subject is a human. The cells may be any cells in the subject (e.g., normal or neoplastic cells), other than microbial cells and leukocytes (i.e., the cells are non-microbial, non-leukocyte cells). A microbe is a microscopic (i.e., too small to be seen with the naked eye) living organism, or microorganism. Examples of microbes include, without limitation, an alga, bacterium, fungus, protozoan, and virus. A leukocyte (leucocyte), or white blood cell, is a nucleated cell (generally, a pale, spherical, and colorless corpuscle or mass), that functions within the immune system by destroying invading cells and removing debris. Examples of leukocytes include, without limitation, polymorphonuclear neutrophils (polymorphs), lymphocytes, eosinophils, and basophils.

The method of the present invention comprises inhibiting CD46 in the subject. CD46 may be inhibited in the subject by any method described herein. By way of example, CD46 may be inhibited in the subject by administering a CD46 inhibitor to the subject. Examples of CD46 inhibitors are described above. Preferably, the CD46 inhibitor is an anti CD46 antibody. The CD46 inhibitor may be provided to the subject in an amount effective to inhibit adhesion of cells in the subject. The effective amount may vary depending upon the desired amount or degree of inhibition of cell-cell adhesion, the subject's weight, severity of the subject's condition, etc. This amount may be readily determined by the skilled artisan, based upon known procedures and methods disclosed herein. In one embodiment, the method of the present invention further comprises the step of inhibiting β1 integrin (e.g., β1 integrin) in the subject.

The development and spread of tumors depends, in part, on the ability of neoplastic cells to adhere to surfaces and/or to each other. Accordingly, the present invention also provides a method for treating or preventing neoplasia in a subject, by inhibiting CD46 in neoplastic cells of the subject. Neoplasias which may be treated/prevented by the present method include morphological irregularities in cells in tissue of a subject, as well as pathologic proliferation of cells in tissue of a subject, as compared with normal proliferation in the same type of tissue. Examples of such neoplasias include, without limitation, carcinomas, particularly those of the bladder, breast, cervix, colon, head, kidney, lung, neck, ovary, prostate, and stomach; malignant melanomas; myeloproliferative diseases; and sarcomas, particularly Ewing's sarcoma, hemangiosarcoma, Kaposi's sarcoma, liposarcoma, peripheral neuroepithelioma, and synovial sarcoma (Beers and Berkow (eds.), The Merck Manual of Diagnosis and Therapy, 17^{th} ed. (Whitehouse Station, N.J.: Merck Research Laboratories. 1999) 973-74, 976, 986, 988, 991).

In accordance with the method of the present invention, CD46 may be inhibited in a subject who has a neoplasia, or who is a candidate for developing a neoplasia, by administering a CD46 inhibitor to the subject, either alone or in combination with one or more antineoplastic drugs used to treat neoplasias. Examples of antineoplastic drugs with which the CD46 inhibitor may be combined include, without limitation, carboplatin, cyclophosphamide, doxorubicin, etoposide, and vincristine.

In the method of the present invention, a CD46 inhibitor is administered to a subject who has neoplasia, or who is a candidate for developing a neoplasia, in an amount effective to treat the neoplasia in the subject. As used herein, the phrase “effective to treat the neoplasia” means effective to ameliorate or minimize the clinical impairment or symptoms resulting from the neoplasia. For example, the clinical impairment or symptoms of the neoplasia may be ameliorated or minimized by diminishing any pain or discomfort suffered by the subject; by extending the survival of the subject beyond that which would otherwise be expected in the absence of such treatment; by inhibiting or preventing the development or spread of the neoplasia; or by limiting, suspending, terminating, or otherwise controlling the maturation and proliferation of cells in the neoplasm. The amount of CD46 inhibitor that is effective to treat neoplasia in a subject will vary depending on the particular factors of each case, including the type of neoplasia, the stage of neoplasia, the subject's weight, the severity of the subject's condition, and the method of administration. These amounts can be readily determined by the skilled artisan.

In view of the above-described methods, the present invention provides use of a CD46 inhibitor in a method for inhibiting adhesion of a cell to a substrate. The present invention also provides use of a CD46 inhibitor in a method for inhibiting cell-cell adhesion between or among non-microbial, non-leukocyte cells in a subject, including use of a CD46 inhibitor in a method for treating or preventing a neoplasia in a subject.

The present invention further provides a method for promoting adhesion of a cell to a substrate. The cell and substrate may be any of those described herein. By way of example, the cell may be a retinal pigment epithelium (RPE) cell. Furthermore, by way of example, the substrate may be a medical device (including a prosthetic device) or an extracellular matrix (e.g., Bruch's membrane).

The method of the present invention comprises increasing CD46 in a cell. CD46 may be increased by activating, facilitating, and/or stimulating the function or activity of CD46 in a cell, tissue, or subject, and/or by increasing the amount, level, or expression of CD46 in a cell, tissue, or subject. The term “activating”, as used herein, means stimulating or inducing a function or activity of CD46 in a cell, particularly its ability to facilitate cell-cell or cell-substrate adhesion. CD46 function/activity and/or level/expression may be increased by targeting CD46 directly, and/or by targeting CD46 in an indirect manner (e.g., by directly or indirectly causing, inducing, or stimulating the upregulation of CD46 activity or expression within a cell or tissue, and/or by targeting an enzyme or other endogenous molecule that regulates or modulates the activity or expression of CD46 in a cell or tissue). Preferably, the activity or level of CD46 in the cell is increased by at least 10% in the method of the present invention. More preferably, CD46 in the cell is increased by at least 20%.

In the method of the present invention, CD46 in a cell may be increased by contacting the cell with a modulator of CD46 (i.e., an agent that modulates CD46 level and/or activity in a cell). The modulator may be any protein, polypeptide, peptide, nucleic acid (including DNA or RNA), antibody, Fab fragment, F(ab′)₂ fragment, molecule, compound, antibiotic, drug, or agent reactive with CD46, that activates CD46 function or activity and/or induces or upregulates CD46 expression. Modulators of CD46 may be identified using a simple screening assay. For example, to screen for candidate modulators of CD46, human RPE cells may be plated onto microtiter plates, then contacted with a library of drugs. Any resulting increase in, or upregulation of, CD46 expression then may be detected using nucleic acid hybridization and/or immunological techniques known in the art, including an ELISA. Additional modulators of CD46 expression may be identified using screening procedures well known in the art or disclosed herein. Modulators of CD46 will be those drugs which induce or upregulate activity and/or expression of CD46. In a similar manner, candidate modulators also may be screened for their ability to promote adhesion between a cell and a substrate (or between/among cells).

In accordance with the method of the present invention, CD46 may be increased in a cell by contacting the cell with a CD46 modulator. The CD46 modulator is provided in an amount that is effective to promote adhesion of a cell to a substrate. This amount may be readily determined by the skilled artisan, based upon known procedures and methods disclosed herein. In one embodiment, the method of the present invention further comprises the step of increasing β integrin (e.g., β1 integrin) in the cell. β integrin may be increased in the cell by contacting the cell with a modulator of β integrin. The β integrin modulator is provided in an amount which, in combination with an effective amount of CD46 modulator, is effective to promote adhesion of a cell to a substrate. This amount may be readily determined by the skilled artisan, based upon known procedures and methods disclosed herein.

In the method of the present invention, cells may be contacted with effective amounts of CD46 and β integrin modulators either in vitro, or in vivo in a subject. The modulators may be contacted with the cells by introducing the modulators into the cells, in accordance with known techniques used for the introduction and administration of proteins, nucleic acids, and other drugs, and/or methods described herein. Alternatively, the modulators may be contacted with cells in vivo in a subject, by introducing the modulators into the subject (e.g., by introducing the modulators into cells of the subject), or by administering the modulators to the subject, in accordance with known techniques and/or methods described herein. Where the modulators are contacted with a cell in vivo, the subject is preferably a human. The cells may be contained in any tissue of a subject (e.g., retinal pigment epithelium, smooth muscle epithelium, etc.), and may be detected in tissue of the subject by standard detection methods readily determined from the known art including, without limitation, immunological techniques (e.g., immunohistochemical staining), fluorescence imaging techniques, and microscopic techniques.

It is expected that the present method will be useful for increasing the adhesion of transplanted biological tissues to their underlying matrices. For example, the present method may be used to facilitate transplantation of RPE cells to a Bruch's membrane. Bruch's membrane is the basement membrane for RPE cells in a normal human eye. It is a five-layered structure located between the highly-fascular choriocapillaris endothelium and the avascular subretinal space. It slowly loses its normal function during age-related macular degeneration (ARMD), and may be damaged during surgery to remove bleeding and blood vessels from under the retina. RPE cell transplantation under the retina is usually unsuccessful, in part, because the sheet of transplanted RPE cells contracts almost immediately after being placed in the subretinal space. As a result, the RPE cells are unable to attach to the surface of Bruch's membrane, and, therefore, undergo apoptosis. It is believed that the present method will increase the ability of RPE cells to resurface a damaged epithelium or a defect in the eye of a patient with ARMD or another related eye disease.

The present invention further provides a method for promoting cell-cell adhesion between or among cells in a subject. The cells may be any cells in the subject, other than microbial cells and leukocytes (i.e., the cells are non-microbial, non-leukocyte cells). The method of the present invention comprises increasing CD46 in the subject. In accordance with the method of the present invention, CD46 may be increased in a subject by contacting cells in the subject with a CD46 modulator and/or by administering a CD46 modulator to the subject. The CD46 modulator is provided in an amount that is effective to promote cell-cell adhesion in the subject. This amount may be readily determined by the skilled artisan, based upon known procedures and methods disclosed herein. In one embodiment, the method of the present invention further comprises the step of increasing β integrin (e.g., β1 integrin) in the cell. β integrin may be increased in the cell by contacting the cell with a modulator of β integrin. The β integrin modulator is provided in an amount which in combination with an effective amount of CD46 modulator, is effective to promote cell-cell adhesion in the subject. This amount may be readily determined by the skilled artisan, based upon known procedures and methods disclosed herein.

The present invention is described in the following Examples, which are set forth to aid in the understanding of the invention, and should not be construed to limit in any way the scope of the invention as defined in the claims which follow thereafter.

### EXAMPLES

### Example 1

### Immunohistochemistry

Posterior globes of donor eyes (ages 5474 years), obtained from the Kentucky Lions Eye Bank, were prepared for immunohistochemistry by fixing in 4% paraformaldehyde overnight, dehydrating, and embedding in paraffin. Consecutive 5-μm sections were hydrated, incubated for 2 h with 0.3% H₂O₂ in phosphate-buffered saline, to quench endogenous peroxidase, and then incubated with 5% goat nor mal serum. The sections were then incubated with 1:100 mouse anti-huma CD46 antibody (BD PharMingen, San Diego, Calif.), washed, and incubated with biotinylated goat anti-mouse antibody, a buffered aqueous solution (ExtrAvidin-HRP; Sigma-Aldrich, St. Louis, Mo.), and a stain (NovaRed Substrate Kit for Peroxidase; Vector Laboratories, Burlingame, Calif.) that produces a red color which contrasts with the gold-brown of the melanin granules in the RPE. Control sections were incubated, either with isotype-matched non-immune serum or without the primary antibody.

The RPE-choroid from one donor eye was prepared as a flat-mount for confocal microscopy and in situ staining with CD46 antibody (as above), and visualized by incubation with a Cy3-conjugated goat anti-mouse antibody (Sigma-Aldrich). RPE cell nuclei were counterstained with 4′,6′-diamino-2-phenylindole (DAPI; Vector Laboratories).

RPE cells, harvested from donor eyes and maintained as primary cultures in DMEM/F12 with 10% fetal bovine serum (FBS), were also prepared for immuno histochemistry and confocal microscopy by fixing in 4% paraformaldehyde for 20 min. Cultured RPE were immunostained for CD46, in the manner just described, and also for the β1 integrin antibody (Chemicon International Temecula, Calif.). The RPE were then visualized by a Cy3-labeled antibody.

Additionally; ARPE19 cells (ATCC, Manassas, Va.), derived from human RPE, were cultured on chamber slides (Nalge Nunc International, Naperville, Ill.) in DMEM F12 containing 10% FBS with 100 U/mL penicillin and 100 μg/in L streptomycin. The cells were maintained in culture conditions for 4 weeks, and were then prepared for CD46 and β1 integrin immunohistochemistry and confocal microscopy, as described above for cultured cells.

All research on tissue obtained from human subjects adhered to the tenets of the Declaration of Helsinki.

### Example 2

### Reverse Transcription Polymerase Chain Reaction

ARPE 19 monolayers maintained in culture for 4 weeks, or confluent RPE cell cultures obtained from human eyes (ages 35-65 years) and established after 26 passages, were used to extract total RNA (RNeasy Mini Kit; Qiagen, Valencia, Calif.), according to the manufacturer's specification. The yield and purity of RNA were estimated by optical density at 260/280 nm. After DNase treatment, cDNAs were synthesized from RNAs with reverse transcriptase (Superscript II; Invitrogen/Gibco, Gaithersburg, Md.), using oligo dT as the primer, according to the manufacturer's specifications. Polymerase chain reactions were performed in an automatic sequencer (GeneAmp PCR System 2400; Applied Biosystems Inc. Foster City, Calif.) with advantage cDNA polymerase mix (Clontech, Palo Alto, Calif.). The following primer pair for CD46 was used: forward, 5′-CCT GCA AAT GGG ACT TAC G3′ (SEQ ID NO:1); reverse, 5′-AAA AAC CCT TATCGC ATT CAA AC3′ (SEQ ID NO:2).

PCR products were sequenced by DNA autosequencing (CEQ 2000; Beckman Instruments, Fullerton, Calif.), and the sequence identity was verified using a BLAST search of the Genome Systems Data Bank, available at the NIH website: http://www.ncbi.nlm.nih. gov/blast/.

### Example 3

### Immunoblot Analysis

To detect expression of the complement regulatory protein, CD46, in RPE, Western-blot analysis was performed with RPE cells from donor eyes (ages 60, 73, and 76 years) and from ARPE19 cell lines. Posterior globes were frozen within 12 h after death, and were stored at 80° C., thawed, and dissected. RPE cells were collected by adding Hanks' medium into the eyecups, and gently pipetting. Total protein extract of RPE cells (collected from donor eyes or ARPE19) was prepared with lysis buffer containing 50 mM Tris HCl (pH 8.0). 1% Triton-X-100, 150 mM NaCl, 0.02% sodium azide, 10 μg/mL antipain, 10 jug/mL leupeptin, 10 μg/mL peptain A, 2 μg/mL aprotinin, and 100 μM phenlylmethylsulfonyl fluoride (PMSF). Protein concentration was determined using a bicinchoninic acid (BCA Protein Assay Reagent Kit; Pierce, Rockford, Ill.). Fifty micrograms of each protein sample were separated by SDS-PAGE on 10% gels under non-reducing conditions. Proteins were then transferred to polyvinylidene difluoride (PVDF) membranes. The membranes were blocked overnight at 4° C. in TBS-T solution containing 5% nonfat dry milk, and incubated with mouse antihuman antibodies against CD46 (BD PharMingen International, San Diego, Calif.) in TBS-T solution. The antibody binding was detected with horseradish-peroxidase-conjugated anti-mouse (Dako, Glostrup, Denmark) secondary antibody, and enhanced chemiluminescence (ECL) Western-blot-detection reagents (Amersham Life Science, Buckinghamshire, UK).

### Example 4

### Immunoprecipitation

RPE cells harvested from human donor eyes or ARPE19 cells were washed with ice-cold PBS, and lysed for 30 mil at 4° C. in non-ionic detergent (Brij 98, Sigma Aldrich; 20 mM Tris (pH 7.5), 150 mM NaCl, 1% Brij 98, 1 mM CaCl₂, 1 mM MgCl₂, 10 μg/mL antipain, 10 μg/mL leupeptin, 10 μg/mL peptain A, 2 μg/mL aprotinin, and 100 μM PMSF). The insoluble material was removed by centrifugation at 12,000 g for 15 min at 4° C., and the supernatants were pre-cleared by incubation with Sepharose protein G (Amersham Pharmacia Biotech) for 1 at 4° C. Pre-cleared supernatants were centrifuged, transferred to new tubes, and then incubated overnight at 4° C. with either anti-CD46 rabbit polyclonal (Santa Cruz Biotechnology, Santa Cruz, Calif.) or anti-β1-integrin monoclonal (Chemicon International) precipitating antibody. The purified rabbit IgG or isotype-matched IgG served as the control. The immune complex was precipitated by adding 1:10 volume of Sepharose protein G, and incubating at 4° C. After a 1-hour incubation under constant rotation, samples were centrifuged aid washed five times with lysis buffer containing 0.5% Brij 98. Bound proteins were eluted with two times lysis buffer and boiling for 10 min. Immunoprecipitated proteins were resolved in a 7.5% SDS gel under non-reducing conditions, and transferred to PVDF membranes. Co-precipitated β1 integrin and CD46 were probed, either by a combination of biotin-labeled mAb (Ancell, Bayport, Minn.) plus horseradish-peroxidase conjugated streptavidin (Amersham Pharmacia Biotech) or a combination of unlabeled mouse mAb (BD PharMingen International) plus HRP-conjugated rabbit anti-mouse secondary antibody (Chemicon International).

### Example 5

### Cell Adhesion Assay

Explants of human Bruch's membrane were prepared from human donor eyes (60 to 70 year-old donors), as previously described (Tezel et al., Fate of human retinal pigment epithelial cells seeded onto layers of human Bruch's membrane. Invest. Ophthalmol. Vis., 40:467-76, 1999; Tezel and Del Priore, Repopulation of different layers of host human Bruch's membrane by retinal pigment epithelial cell grafts. Invest. Ophthalmol. Vis. Sci., 40:767-74, 1999; Del Priore and Tezel, Reattachment rate of human retinal pigment epithelium to layers of human Bruch's membrane. Arch. Ophthalmol., 116:335-41, 1998). After a full-thickness circumferential incision was made posterior to the ora serrata, and the vitreous and anterior segments were removed, the posterior poles were inspected and then discarded if there was any evidence of subretinal blood, drusen, or irregular pigmentation of the macular RPE. Once the neural retina was removed, 0.02 N ammonium hydroxide was pipetted into the eyecup to remove adherent RPE, and was followed by washing with PBS three times. A 6.5 mm diameter corneoscleral trephine was used to punch out explants of human Bruch's membrane from the macula and periphery of the eyecups. Six to eight explants were harvested per eye.

Second passage human RPE cells were harvested from donor eyes (53 to 65 year-old donors) by incubating in 0.25% trypsin/0.25% edetic acid, in Hanks' balanced salt solution, for 20 mill. Ten milliliters of MEM/15 were added for quenching, and the cell suspension was centrifuged for 5 min. at 800 revolutions per minute. The cell pellet was incubated on a shaker table alt room temperature for 1 h, in one of the following antibodies: 0.1-25 μg/mL of mouse anti-human CD46 monoclonal antibody (Accurate Chemical & Scientific Corp., New York, N.Y.), 10 g/mL of mouse anti-human-β1-integrin monoclonal antibody (Chemicon International), or 1:500 mouse nonspecific IgG1κ MOPC21 monoclonal antibody (Sigma-Aldrich). The cell pellet was washed three times, and resuspended in MEM without serum. Aliquots of 5×10⁴ viable cells were then applied to each Bruch's membrane explant. Thereafter, cells were allowed to attach for 24 h. Unattached cells were removed by picking up the tissue with fine forceps, dipping three times in Hanks' balanced salt solution, and then placing the explants in a new well of a 96-well plate. Three explant buttons were used for each experimental condition. Data were analyzed by one-way analysis of variance, followed by a multiple comparison test or a two-tailed t-test.

Cell adhesion assays were performed using an established MTT-based cell assay, as described previously (Ho and Del Priore, Reattachment of cultured human retinal pigment epithelium to extracellular matrix and human Bruch's membrane. Invest. Ophthalmol. Vis. Sci., 38:1110-18, 1997; Mossmann, T., Rapid colorimetric assay for cellular growth and survival: application to proliferation and cytotoxicity assays. J. Immunol. Methods, 65:55-63, 1983). MTT (3(4,5-dimethylthiazol-2-yl)2,5-diphenyltetrazolium bromide; Sigma-Aldrich) is a dye with characteristics that change when it is dehydrogenated by cellular mitochondrial dehydrogenase, and when the activity of this enzyme is proportional to the number of live cells exposed to the dye (Tezel et al., Fate of human retinal pigment epithelial cells seeded onto layers of human Bruch's membrane. Invest Ophthalmol. Vis. Sci., 40:467-76, 1999). The amount of yellow-reduced tetrazolium was quantified with an enzyme-linked immunosorbent assay reader with a 570 nm filter, following removal of solid tissue from the wells containing explants and reading of the 96 well plates. The number of cells attached to the surface was calculated by comparing the enzyme-linked immunosorbent assay readings, obtained on the wells with an unknown number of cells, with a standardized curve, obtained using control wells containing up to 50,000 live cells as an internal standard for each experiment. The attachment proportion was defined and calculated as the proportion of cells that had attached to the Surface during the experimental period. Cell adhesion experiments were performed in triplicate seven times.

Discussed below are results obtained by the inventors in connection with the experiments of Examples 1-5:

Immunolocalization and Expression of CD46

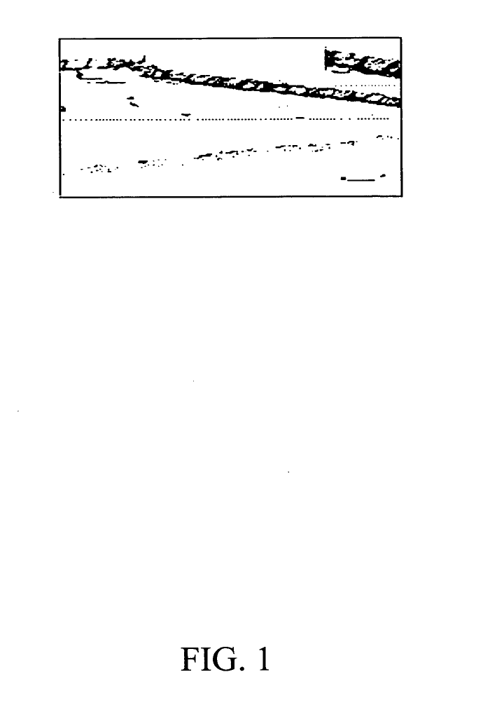

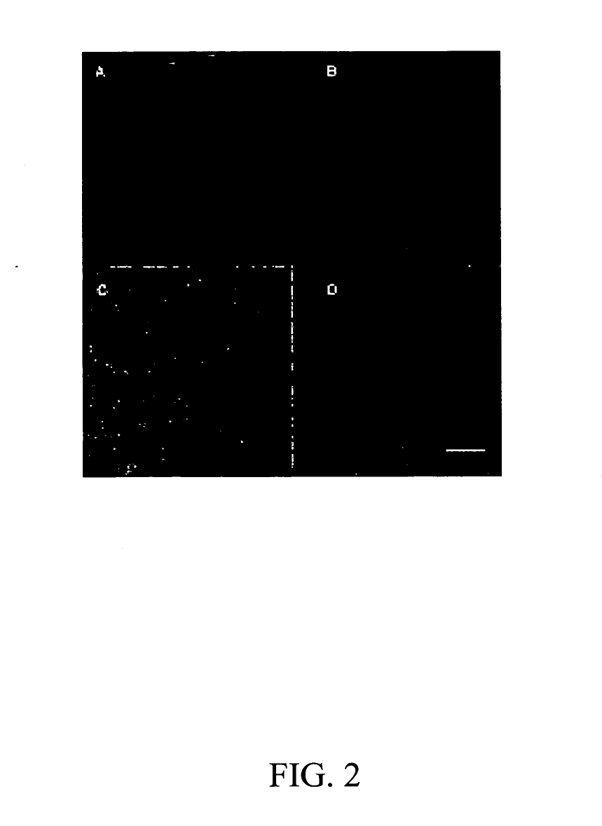

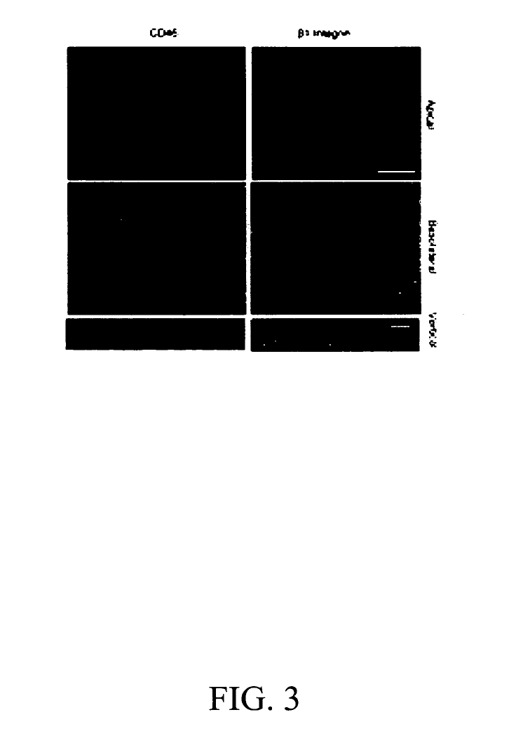

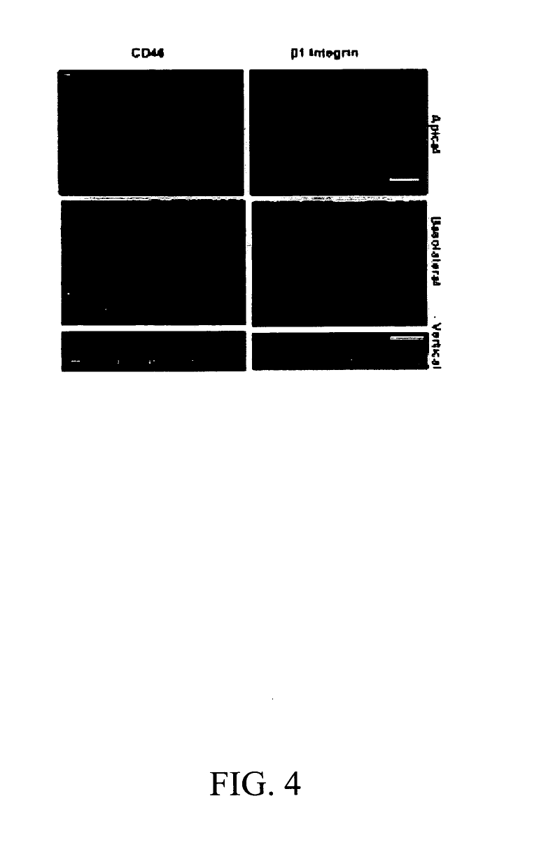

In histologic sections of the normal human eye, CD46 staining showed a preferential distribution on the basolateral surface of the RPE, but not in Bruch's membrane (FIG. 1). Con focal microscopy of horizontal sections of RPE, harvested directly from human donor eyes and prepared as a flat-mount, demonstrated a complete absence of CD46 staining on the apical RPE membranes in situ (FIG. 2A), and an absence of autofluorescent pigment granules (FIG. 2B). CD46 staining was present in sections of the basolateral RPE surface (FIG. 2C): some autofluorescence was present, due to pigment granules in the basal RPE cytoplasm (FIG. 2D). Confocal microscopy of cultures immunolabeled for CD46 and β1 integrin also revealed a basolateral membrane localization of both proteins in horizontal and vertical views of the RPE monolayer—in primary cultured RPE from donor eyes (FIG. 3) and in RPE cell lines (FIG. 4). When the RPE monolayer was viewed at different confocal planes from apical to basolateral surfaces, immunolabeled CD46 and β1 integrin were present only on the basolateral membrane surfaces. When viewed in vertical sections, antibody staining for CD46 and μ1 integrin clearly labeled the basal RPE surface.

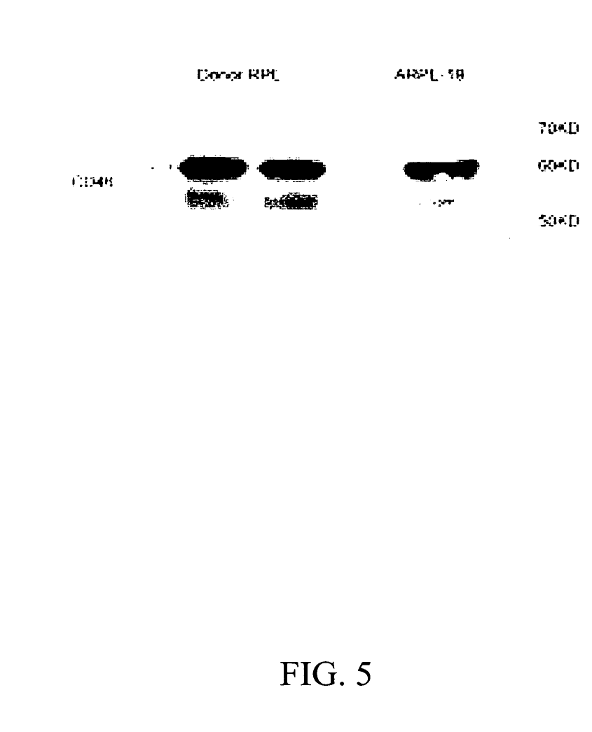

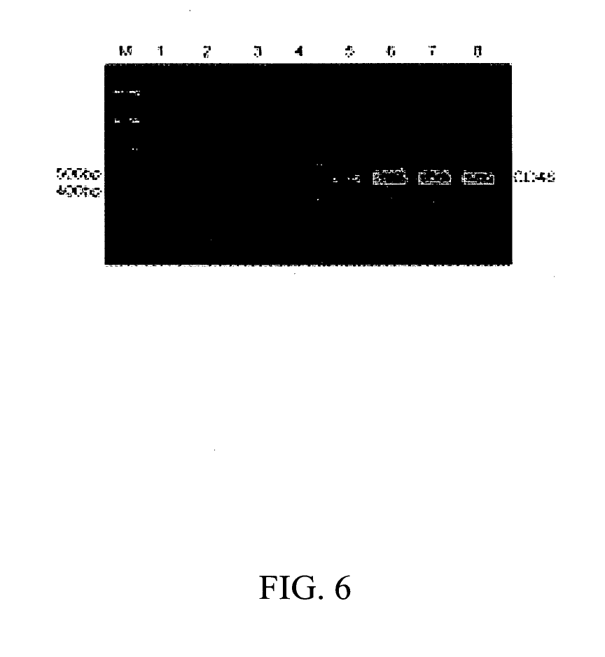

Immunoblot analysis of RPE obtained from donor eyes and the ARPE19 cell line demonstrated the presence of a protein doublet at 55 and 65 kDa, corresponding to the lighter and heavier isoforms of CD46, respectively (FIG. 5). Primary RPE cultures established from donor eyes, and from cultured ARPE19 cells grown to confluence and maintained for 4 weeks or longer, demonstrated the presence of mRNA for CD46 (FIG. 6). A PCR product was obtained in the expected base-pair range of 448 bp for CD46, and sequencing confirmed a 99% sequence identity with its appropriate cDNA.

Co-Immunoprecipitation of CD46 and β1 Integrin in RPE Cells

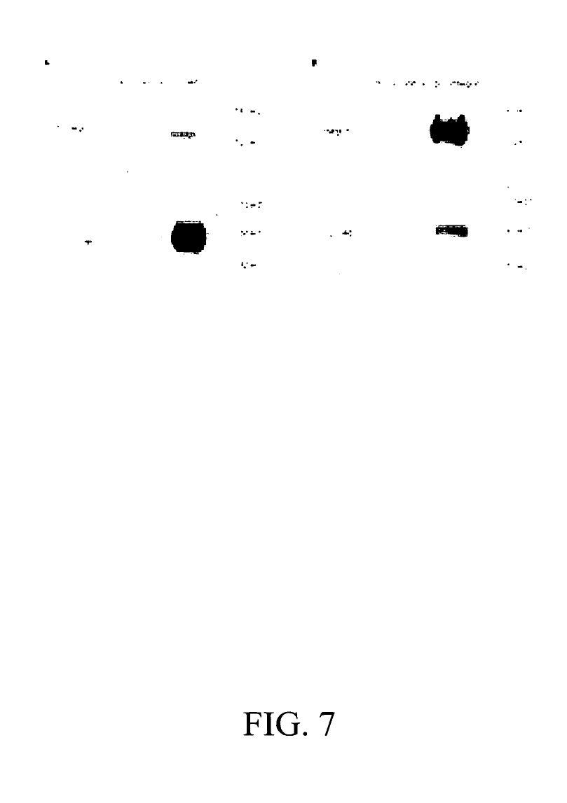

To characterize the association of CD46 with β1 integrin, immunoprecipitation experiments were performed on cell lysates of RPE that had been harvested from human donor eyes and the ARPE19 cell line, and immunoblotted with antibodies to CD46 and β1 integrin. β1 integrin co-precipitated with CD46 from both human RPE (FIG. 7A) and RPE cell lines. Reciprocally, CD46 co-immunoprecipitated with β1 integrin in both human RPE (FIG. 7B) and ARPE19 cell lysates. Control samples incubated with purified rabbit IgG or isotype-matched IgG failed to precipitate either CD46 or β1 integrin.

RPE Cell Adhesion Assay

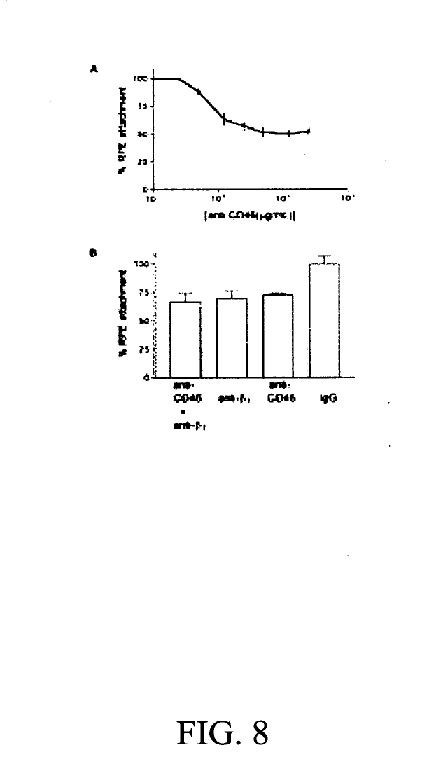

The inventors used the previously-described cell adhesion assay, in which RPE attachment to the native physiological substrate—the basal lamina of human Bruch's membrane—was determined (FIG. 8). RPE cells that were preincubated with an irrelevant IgG (mouse IgG1κ MOPC21 monoclonal antibody), before seeding onto Bruch's membrane, served as the control for all of the adhesion studies. Anti-CD46 antibody reduced RPE adhesion to human Bruch's membrane by approximately 4050%, in a dose-dependent manner, as compared with the control (FIG. 8A); half-maximum inhibition occurred at a concentration of ˜1 μg/l mL of anti-CD46. Previously, the inventors demonstrated that anti β1-integrin antibodies decrease RPE adhesion to human Bruch's membrane and RPE-derived extracellular matrix (Ho and Del Priore, Reattachment of cultured human retinal pigment epithelium to extracellular matrix and human Bruch's membrane. Invest. Ophthalmol. Vis. Sci. 38:1110-18, 1997). Herein, the inventors have shown that 10 μg/mL of anti-β1-integrin, previously shown to be a saturating dose, produced inhibition comparable to anti-CD46 alone; incubation with both anti-CD46 and anti-β1-integrin did not produce further inhibition, when compared with either antibody alone (FIG. 8B).

While the foregoing invention has been described in some detail for purposes of clarity and understanding, it will be appreciated by one skilled in the art, from a reading of the disclosure, that various changes in form and detail can be made without departing from the true scope of the invention in the appended claims.

1. A medical device for use in inhibiting adhesion of cells, wherein the medical device has a coating comprising an inhibitor of CD46.

2. The medical device of claim 1, wherein the coating further comprises an inhibitor of β integrin.

3. Use of a medical device in a method for inhibiting adhesion of cells, wherein the medical device has a coating comprising an inhibitor of CD46.

4. A method for inhibiting adhesion of a cell to a substrate, comprising inhibiting CD46 in the cell.

5. The method of claim 4, further comprising the step of inhibiting β integrin in the cell.

6. Use of a CD46 inhibitor in a method for inhibiting adhesion of a cell to a substrate.

7. A method for inhibiting cell-cell adhesion between or among non-microbial, non-leukocyte cells in a subject, comprising inhibiting CD46 in the subject.

8. The method of claim 7, further comprising the step of inhibiting β integrin in the subject.

9. Use of a CD46 inhibitor in a method for inhibiting cell-cell adhesion between or among non-microbial, non-leukocyte cells in a subject.

10. A method for promoting adhesion of a cell to a substrate, comprising increasing CD46 in the cell.

11. The method of claim 10, wherein the method further comprises increasing β integrin in the cell.

12. A method for promoting cell-cell adhesion between or among non-microbial, non-leukocyte cells in a subject, comprising increasing CD46 in the subject.

13. The method of claim 12, wherein the method further comprises increasing β integrin in the subject.
